SEQUENCE LISTING (1 sequence)

SEQ ID NO: 1 - protein, 11 aa, Artificial Sequence
  synthetic peptide not based on any known sequence of natural origin
TASGVAETTNC

(19) United States
(12) Patent Application Publication
(10) Pub. No.: US 2006/0068446 A1
(43) Pub. Date: Mar. 30, 2006

(54) Methods

(75) Inventors: Shiranee Sriskandan, London (GB); Daniel Altmann, London (GB)

Correspondence Address: NIKOLAI & MERSEREAU, P.A., 900 SECOND AVENUE SOUTH, SUITE 820, MINNEAPOLIS, MN, 55402, US

(73) Assignee: Imperial College Innovations Limited, London (GB)

(21) Appl. No.: 11/223,252

(22) Filed: Sep. 7, 2005

Related U.S. Application Data
(63) Continuation of application No. PCT/GB04/01004, filed on Mar. 8, 2004.

(30) Foreign Application Priority Data
Mar. 7, 2003 (GB) 0305252.9

(51) Int. Cl. G01N 33/567; G01N 33/53

(52) U.S. Cl. 435/7.2

(57) ABSTRACT

A method for identifying a compound for modulating the immune response in a first type of mammal to a super-antigen, comprising the steps of 5 (7) providing a cell expressing a MHC Class II molecule derivable at least in part from the first type, or cells comprising such a cell or cells (8) exposing the cell or cells provided in step (1) to a test compound (9) before, after or during step (2) exposing the cell or cells to the superantigen (10) determining whether the test compound modulates a response of the cell or cells to exposure to the superantigen (11) determining whether the test compound modulates a response of such a cell or cells to exposure to a conventional antigen, and (12) selecting a compound which modulates a response to the superantigen but does not modulate the response to exposure to the conventional antigen. The test compound may be a monoclonal antibody reactive with MHC Class II molecules. A method of treating an individual with or at risk of superantigen exposure/poisoning comprising administering to the individual the antibody TDR3 1.1 or molecule having the binding specificity of TDR31.1.

The present invention relates to methods of modulating the immune response to superantigens; and methods for identifying compounds for modulating the immune response to superantigens.

The term superantigen (SAG) was introduced in 1990 by Marrack & Kappler (Science 248, 705-711) to describe toxins that lead to excessive expansion of CD4⁺ T cells displaying specific β-chain variable regions (Vβ) of the T-cell antigen receptor (TCR). Conventional antigens undergo processing by antigen presenting cells and the fragments are presented to the TCR when bound in the antigen-binding groove of major histocompatibility complex (MHC) Group II molecules on the surface of the antigen presenting cells. Recognition by all five variable elements (Vβ, Dβ, Jβ, Vα, Jα) forming the hypervariable recognition loops of the TCR is then required in order for stimulation of the T cell. Conventional antigens stimulate in the order of 1 in 10,000 T cells. In contrast, SAGs bind directly to the MHC Group II molecules on the surface of antigen presenting cells without being processed. They bind outside the normal antigen-binding groove on the MHC Class II molecules and the subsequent interaction with TCRs depends only on the Vβ chain of the TCR rather than the entire hypervariable region. As a consequence of bypassing the specificity of the TCR, SAGs can stimulate over 20% of all T cells. This in turn leads to massive release of host cytokines, which are believed to be responsible for the toxic effects of the SAGs, which include capillary leakage leading to hypotension, shock, multiorgan failure and death. Consistent with their ability potentially to stimulate T cells specific for self-antigens, SAGs are also thought to have a possible role in autoimmune diseases and other abnormal immunologic states such as psoriasis, atopic dermatitis and Kawasaki syndrome. SAGs and their involvement in toxic shock syndrome and other medical conditions are reviewed in, for example, McCormick et al (2001) Ann Rev Microbiol 55, 77-104.

SAGs include the staphylococcal toxin toxic shock syndrome toxin-1 (TSST-1), staphylococcal enterotoxins (SE) A, B, Cₙ, D, E, F, H, I, J, K, L, P (reviewed in Dinges et al (2000) Clin Microbiol Rev 13, 16-34), streptococcal pyrogenic exotoxins (SPE) A, C, G, H, J, streptococcal superantigen and multiple variants of streptococcal mitogenic exotoxin Z (SMEZₙ) (reviewed in Kotb (1995) Clin Microbiol Rev 8, 411-426; McCormic & Schlievert (2000) in Gram-positive Pathogens Fischetti et al eds, Americal Soc Microbiol, Washington DC; pp 43-52; see also Unnikrishnan et al (2002) J Immunol 169, 2561-2569. TSST-1 is believed to be responsible for nearly all cases of menstrual-associated TSS. SEB and SEC are considered to be responsible for most cases of non-menstrual TSS. SEA through to SEE are also common causes of food poisoning.

SAGs have been proposed as targeted therapeutic agents. The SAG may be mutated in order to reduce its seroreactivity whilst retaining SAG function (see, for example, WO 03/002143). Modified SAGs or SAG fragments have also been proposed as therapeutic agents for manipulating the immune system, for example in autoimmune diseases; see for example U.S. Pat. No. 5,859,207.

SAGs are also important in the field of biowarfare and bioterrorism. For example, the staphylococcal enterotoxins have been identified as potential biowarfare/bioterrorism agents. They can cause severe emesis and diarrhea if ingested and in primates can lead to respiratory distress and circulatory collapse if inhaled. They are easy to clone and express, are active at femtomolar-picomolar amounts in vitro and can be lethal at microgram quantities in vivo.

Accordingly, there is a need for methods for treating or preventing superantigen-linked poisoning or disease.

There has been much focus on the role of the T cell as the “effector cell” in superantigen disease. Work on potential anti-superantigen therapies has focussed on blocking interaction with the T cell receptor (see for example Kieke et al (2001) J Mol Biol 307, 1305-1315) or vaccination using fragments of superantigens or modified superantigens that are considered to have reduced T cell and/or MHC Class II binding (see for example U.S. Pat. No. 6,399,332; WO 00/09154; WO 96/14744; US 2003/0009015); or a sequestering/competition effect using a receptor mimic (see Lehnert et al (2001) Biochemistry 40, 4222-4228).

Visvanathan et al (2001) Infection and Immunity 69, 875-884 describes an inhibitory peptide selected from regions of superantigens considered to have highly conserved sequence homology. This is suggested to be able to bind to human MHC Class II molecules. The relevance of this is suggested to be an ability to block binding of the superantigen to the Class II molecules. No evidence is presented to suggest that this is the mode of action on cells or in vivo and it appears that the peptide's effect in vivo (in a system in which human Class II molecules are not present) is due to a vaccine effect.

We have surprisingly found that a molecule that binds to MHC class II molecules at a site which is believed to be distinct from the site at which a superantigen interacts with the MHC class II molecules is able to modulate the immune response to the superantigen, whilst response to a conventional antigen was unaffected. In particular, a monoclonal antibody reactive with human Class II molecules is able to modulate the immune response to a superantigen. Other tested monoclonal antibodies also reactive with human Class II molecules did not have this effect. The monoclonal antibody is considered to bind to a different part of the Class II molecule to the part to which the superantigen binds. Further, the monoclonal antibody's effect on the immune response to the superantigen was directed to the superantigen; the response to a non-superantigen was not affected. The monoclonal antibody does not produce a general immunosuppressive effect.

Accordingly, we consider that it is possible for a molecule to be able to block superantigen signalling rather than merely competing with the superantigen for binding to the Class II molecules. A molecule affecting MHC Class II signalling may be particularly useful in the context of biowarfare/bioterroism where mucosal exposure and class II interactions may be of key imprtance, and where a rapid (but not necessarily long-lasting) response (ie unlike a response produced by vaccination) is required. We have found that superantigen effects on human class II positive cells can be independent of T cell receptor signalling, using mutants which have specified defects in either class II binding or T cell receptor binding. Work on mutated toxins which do not cause T cell proliferation, but which kill rabbits (Analysis of toxicity of streptococcal pyrogenic exo toxin A mutants Roggiani et al. Infect Immun 1997; 65:2868-2875 and Structures of five mutants of toxic shock syndrome toxin-1 with reduced biological activity Earhart et al; Biochemistry 1998; 37: 7194-7202) is also consistent with MHC class II interactions being of key importance.

A first aspect of the invention provides a method for identifying a compound for modulating the immune response in a first type of mammal to a superantigen, comprising the steps of
(1) providing a cell expressing an MHC Class II molecule derivable at least in part from the first type of mammal, or cells comprising such a cell or cells
(2) exposing the cell or cells provided in step (1) to a test compound (3) before, after or during step (2) exposing the cell or cells to the superantigen
(4) determining whether the test compound modulates a response of the cell or cells to exposure to the superantigen
(5) optionally, determining whether the test compound modulates a response of such a cell or cells to exposure to a conventional antigen, and
(6) selecting a compound which modulates a response to the superantigen and does not modulate the response to exposure to the conventional antigen.

Step 5 may be performed on all test compounds or may be performed only on test compounds that modulate a response to the superantigen. Thus, for example, the test of step 5 may be performed for all test compounds in parallel with the test for modulation of a response to a superantigen; or may be performed once the results of the test for modulation of a response to a superantigen is known (and possibly also after other tests have been performed, for example tests in whole animals), so that only compounds of potential interest are assessed in this manner.

The first type of mammal is typically a primate, for example a human. Toxic shock and superantigen-induced emesis are primarily effects seen in primates of exposure to SAGs.

The test compound may be an antibody (preferably a monoclonal antibody) reactive with an MHC Class II molecule, or a molecule having the binding specificity of an antibody reactive with an MHC class II molecule. For example, the molecule may be a recombinant antibody or antibody fragment retaining the binding specificity of a natural antibody (including an unmodified monoclonal antibody). Such an antibody or antibody fragment may retain the variable regions of the original antibody but may not necessarily retain the remainder of the structure of the original antibody. In an alternative approach, the epitope to which the (preferably monoclonal) antibody binds is mapped using known techniques (for example by incubation of monoclonal antibody or antibodies with peptides spanning the relevant class II structure, using a robotic peptide synthesizer; or using known techniques for identifying epitopes or mimotopes, for example using an “epitope library”; see, for example, Scott & Smith (1990) Science 249, 386-390 and WO 97/18236 and references therein). The identified epitope/amino acid sequences may then be used to design or select small molecules (which may be a peptide or peptidomimetic compound) which would be predicted to bind to the identical region of the class II structure. Molecules may be selected for binding to the identified epitope/amino acid sequence/mimotope by affinity screening assays, as well known to those skilled in the art, for example using phage display techniques or array-based binding techniques.

The display of proteins and polypeptides on the surface of bacteriophage (phage), fused to one of the phage coat proteins, provides a powerful tool for the selection of specific ligands. This ‘phage display’ technique was originally used by Smith in 1985 (Science 228, 1315-7) to create large libraries of antibodies for the purpose of selecting those with high affinity for a particular antigen. More recently, the method has been employed to present peptides, domains of proteins and intact proteins at the surface of phages in order to identify ligands having desired properties.

The principles behind phage display technology are as follows:
(i) Nucleic acid encoding the protein or polypeptide for display is cloned into a phage;
(ii) The cloned nucleic acid is expressed fused to the coat-anchoring part of one of the phage coat proteins (typically the p3 or p8 coat proteins in the case of filamentous phage), such that the foreign protein or polypeptide is displayed on the surface of the phage;
(iii) The phage displaying the protein or polypeptide with the desired properties is then selected (e.g. by affinity chromatography) thereby providing a genotype (linked to a phenotype) that can be sequenced, multiplied and transferred to other expression systems.

Alternatively, the foreign protein or polypeptide may be expressed using a phagemid vector (i.e. a vector comprising origins of replication derived from a phage and a plasmid) that can be packaged as a single stranded nucleic acid in a bacteriophage coat. When phagemid vectors are employed, a “helper phage” is used to supply the functions of replication and packaging of the phagemid nucleic acid. The resulting phage will express both the wild type coat protein (encoded by the helper phage) and the modified coat protein (encoded by the phagemid), whereas only the modified coat protein is expressed when a phage vector is used.

Methods of selecting phage expressing a protein or peptide with a desired specificity are known in the art. For example, a widely used method is “panning”, in which phage stocks displaying ligands are exposed to solid phase coupled target molecules, e.g. using affinity chromatography.

Alternative methods of selecting phage of interest include SAP (Selection and Amplification of Phages; as described in WO 95/16027) and SIP (Selectively-Infective Phage; EP 614989A, WO 99/07842), which employ selection based on the amplification of phages in which the displayed ligand specifically binds to a ligand binder. In one embodiment of the SAP method, this is achieved by using non-infectious phage and connecting the ligand binder of interest to the N-terminal part of p3. Thus, if the ligand binder specifically binds to the displayed ligand, the otherwise non-infective ligand-expressing phage is provided with the parts of p3 needed for infection. Since this interaction is reversible, selection can then be based on kinetic parameters (see Duenas et al., 1996, Mol. Immunol. 33, 279-285).

The use of phage display to isolate ligands that bind biologically relevant molecules has been reviewed in Felici et al. (1995) Biotechnol. Annual Rev. 1, 149-183, Katz (1997) Annual Rev. Biophys. Biomol. Struct. 26, 27-45 and Hoogenboom et al. (1998) Immunotechnology 4(1), 1-20. Several randomised combinatorial peptide libraries have been constructed to select for polypeptides that bind different targets, e.g. cell surface receptors or DNA (reviewed by Kay, 1995, Perspect. Drug Discovery Des. 2, 251-268; Kay and Paul, 1996, Mol. Divers. 1, 139-140). Proteins and multimeric proteins have been successfully phage-displayed as functional molecules (see EP 0349578A, EP 0527839A, EP 0589877A; Chiswell and McCafferty, 1992, Trends Biotechnol. 10, 80-84). In addition, functional antibody fragments (e.g. Fab, single chain Fv [scFv]) have been expressed (McCafferty et al., 1990, Nature 348, 552-554; Barbas et al., 1991, Proc. Natl. Acad. Sci. USA 88, 7978-7982; Clackson et al., 1991, Nature 352, 624-628), and some of the shortcomings of human monoclonal antibody technology have been superseded since human high affinity antibody fragments have been isolated (Marks et al., 1991, J. Mol. Biol. 222, 581-597; Hoogenboom and Winter, 1992, J. Mol. Biol. 227, 381-388). Further information on the principles and practice of phage display is provided in Phage display of peptides and proteins: a laboratory manual Ed Kay, Winter and McCafferty (1996) Academic Press, Inc ISBN 0-12-402380-0, the disclosure of which is incorporated herein by reference.

The term “peptidomimetic” refers to a compound that mimics the conformation and desirable features of a particular peptide as a therapeutic agent, but that avoids the undesirable features. For example, morphine is a compound which can be orally administered, and which is a peptidomimetic of the peptide endorphin.

There are a number of different approaches to the design and synthesis of peptidomimetics. In one approach, such as disclosed by Sherman and Spatola, J. Am. Chem. Soc., 112: 433 (1990), one or more amide bonds have been replaced in an essentially isoteric manner by a variety of chemical functional groups. This stepwise approach has met with some success in that active analogues have been obtained. In some instances, these analogues have been shown to possess longer biological half-lives than their naturally-occuring counterparts. Nevertheless, this approach has limitations. Successful replacement of more than one amide bond has been rare. Consequently, the resulting analogues have remained susceptible to enzymatic inactivation elsewhere in the molecule. When replacing the peptide bond it is preferred that the new linker moiety has substantially the same charge distribution and substantially the same planarity as a peptide bond.

Retro-inverso peptidomimetics, in which the peptide bonds are reversed, can be synthesised by methods known in the art, for example such as those described in Mézière et al (1997) J. Immunol. 159 3230-3237. This approach involves making pseudopeptides containing changes involving the backbone, and not the orientation of side chains. Retro-inverse peptides, which contain NH—CO bonds instead of CO—NH peptide bonds, are much more resistant to proteolysis.

In another approach, a variety of uncoded or modified amino acids such as D-amino acids and N-methyl amino acids have been used to modify mammalian peptides. Alternatively, a presumed bioactive conformation has been stabilised by a covalent modification, such as cyclisation or by incorporation of γ-lactam or other types of bridges. See, eg. Veber et al, Proc. Natl. Acad. Sci. USA, 75:2636 (1978) and Thursell et al, Biochem. Biophys. Res. Comm., 111:166 (1983).

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

One approach to the synthesis of cyclic stabilised peptidomimetics is ring closing metathesis (RCM). This method involves steps of synthesising a peptide precursor and contacting it with a RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), has been to design peptide mimics through the application of the transition state analogue concept in enzyme inhibitor design. For example, it is known that the secondary alcohol of staline mimics the tetrahedral transition state of the scissile amide bond of the pepsin substrate. However, the transition state analogue concept has no apparent relevance to hormone agonist/antagonist design or antibody/binding site design.

The test compound may be a drug-like compound or lead compound for the development of a drug-like compound. The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may have a molecular weight of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

In an embodiment, the test antibody or molecule is selected as reactive with the type of MHC Class II molecule expressed by the cell, particularly the part derivable from the first type of mammal, for example human. Thus, in an embodiment, the test antibody or molecule is one considered to bind to a human MHC Class II molecule or molecules, for example an antibody or molecule considered to bind to one or more HLA-DR molecules and/or one or more HLA-DQ molecules, and/or one or more HLA-DP molecules. It is considered that an antibody or molecule able to bind HLA-DR molecules (preferably all or nearly all such molecules) or HLA-DQ molecules (preferably all or nearly all such molecules) is a particularly suitable test compound as SAGs are considered to bind predominantly to HLA-DR or HLA-DQ molecules.

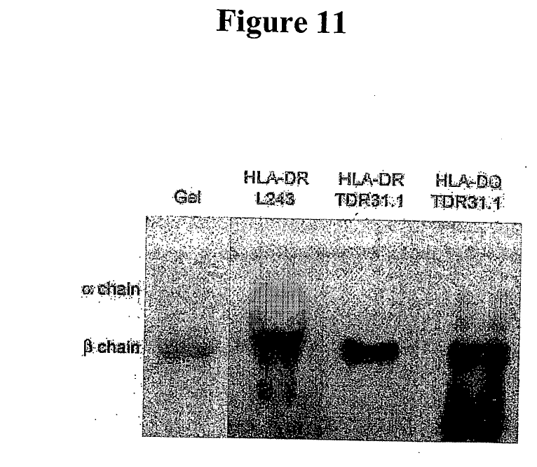

The test compound may be a compound that is considered to share the binding specificity of monoclonal antibody TDR31.1, a monoclonal antibody identified in Example 1 and further Examples as able to modulate the immune response to SAG and further superantigens. TDR31.1 is considered to bind to HLA-DR, HLA-DQ and HLA-DP MHC Class II β chain molecules. See DeKrester et al (1982) Eur J Immunol 12, 214-221; Busch & Rothbard (1990) J Immunol Methods 134, 1-22; Wacherpfennig & Strominger (1995) J Exp Med 181, 1597-1601; Altmann et al (1990) Immunogenetics 32(1), 51 and FIG. 11. TDR31.1 is available from, for example, Ancell Corporation (Bayport, Minn. 5503 USA; product No ANC-131), ID Labs Inc (PO Box 1145 Station B, London, ON, Canada) and Alexis Biochemicals (Q-Biogene-Alexis Ltd, PO Box 6757, Bingham, Nottingham, NG13 8LS).

As is well known to those skilled in the art, the variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299. Chimaeric antibodies are discussed by Neuberger et al (1988, 8th International Biotechnology Symposium Part 2, 792-799). Suitably prepared non-human antibodies can be “humanized” in known ways, for example by inserting the CDR regions of mouse antibodies into the framework of human antibodies.

By “ScFv molecules” we mean molecules wherein the V_{H} and V_{L} partner domains are linked via a flexible oligopeptide.

The advantages of using antibody fragments, rather than whole antibodies, may be several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites. Monovalent molecules may be useful in relation to the present invention, though bivalency may be beneficial in some cases.

As is also well known to those skilled in the art, MHC Class II molecules found in different mammals are similar but not identical. Such differences (as with other differences in immune system components) may be particularly important in relation to response to SAGs. For example, humans are much more susceptible to most SAGs than are mice. Mice are in general poor models for assessing response to SAGs, requiring the use of presensitising agent such as lipopolysaccharide and D-galactosamine (which disrupts liver metabolism); see, for example Welcher et al (2002) J Infect Dis 186, 501-510 for a discussion. However, testing in humans or primates is clearly undesirable.

Accordingly, in an embodiment, the cell expressing a MHC Class II molecule derivable at least in part from the first type of mammal is a cell of a second type of mammal which expresses at least part of an MHC Class II molecule of the first type of mammal. In particular, the second type of mammal may be a laboratory animal, for example a laboratory rodent such as a rabbit, rat or mouse. In an embodiment, the second type of mammal is a mouse. Larger animals, for example pig, may also be useful for further tests; for example larger animals may be able to vomit, thereby allowing tests of efficacy against this response.

The first type of mammal may be any type of mammal for which it is desired to identify an agent useful in preventing or treating superantigen exposure/poisoning. The first type of mammal may be a human. Alternatively, the first type of mammal may be an agricultural, domestic or military animal, for example a horse or dog.

As well known to those skilled in the art, MHC Class II molecules have two types of constituent polypeptide chains; an α chain and a β chain. Human MHC Class II molecules fall into three groups:. HLA-DR, HLA-DQ and HLA-DP, each of which is highly polymorphic. Thus, within each of these groups there are multiple alleles. Some antigenic determinants are shared by molecules of all three groups, whereas others are shared by members of one group only, and still others are characteristic of a particular allele. HLA genes are reviewed in, for example, Klein et al (1993) Sci Am 269, 78-83; see also WO 95/14377. See also Kimura & Sasazuki (1992) 11 International Histocompatibility Workshop reference protocol for the HLA DNA typing technique, p397-419, in K Tsuji, M Aizawa and T Sasazuki (ed), HLA 1991, Oxford University Press, Oxford, United Kingdom or Nevinny-Sticke et al (1991) Nonradiactive HLA class II typing using polymerase chain reaction and digoxigenin-11-2′-3′-dideoxy-uridine-triphosphate labeled oligonucleotide probes. Hum Immunol 31, 7-13. HLA-DR, DQ, DP nomenclature is reviewed in Marsh S G: Tissue Antigens 2002 December; 60(6):544-5. The mouse homologues of HLA-DR and HLA-DQ are H2-E and H2-A respectively (also known as I-E and I-A)

Staphylococcal SAGs are considered to bind prefererentially to HLA-DR molecules (though not all binding at the same place on those molecules) whereas SPEA and certain other streptococcal SAGs bind preferentially to HLA-DQ molecules (see, for example Welcher et al (2002) J Infect Des 186, 501-510; Sriskandan et al (2001) J Infect Dis 184, 166-173; and references cited either therein).

Examples of transgenic models which may be useful as whole animals for whole animal in vivo testing, or as sources of cells for cell based screens include those described in the following documents:
WO 95/14377—transgenic mouse models; particularly human CD4+, mouse CD4−/−, mouse CD8 −/−, human DQw6+ (hCD4+, mCD4−/−, mCD8−/). Other models are also referred to on pages 5 to 6; for example Nishimura et al (1990) J Immunol 145, 353-360 describing a mouse carrying the transgenes encoding the alpha and beta chains of the human HLA-DQw6 protein).
Sriskandan et al (2001) J Infect Dis 184, 166-173 and Unnikrishnan et al (2002) J Immunol 169, 2561-2569 and references therein—C57BL/10.DQ8 (carrying genomic constructs for DQA1*0301 and DQB*0302) and FVB/N.DR1 (carrying genomic constructs for DRA1*0101 and DRB1*0101)
Welcher et al (2002) J Infect Des 186, 501-510 and DaSilva et al (2002) J Infect Dis 185, 1754-1760 and references therein—HLA Class II transgenic mice (HLA-DR2 (HLA-DR2β with murine IEα), HLA-DR3 and HLA-DQ8/human CD4⁺) on a MHC Class II knockout (Aβ°) background

These documents, particularly WO 95/14377 also provide details on how further transgenic animals may be prepared.

Example 1 further describes HLA-DR1 and HLA-DR15 transgenic models. The DR1 transgenic mice are also described in Altmann et al The T cell response of HLA-DR transgenic mice to human myelin basic protein and other antigens in the presence and absence of human CD4 J Exp med 1995; 181: 867-875. The mice used in Example 1 are not knock-outs for the mice MHC class II molecules or for mouse CD4 or CD8.

It will be clear from the above that it may not be necessary for there to be a knock-out of the host's MHC class II genes, or of other relevant immune system molecules such as CD4 or CD8. Neither may it be necessary for the “target” organism's CD4 gene to be present in addition to the MHC class II α chain and/or β chain genes. In general, for the present invention it is desirable for the host's MHC class II genes not to be knocked out. This may facilitate examining whether a normal immune response is retained. It is considered that the normal immune response may go ahead through the host (for example mouse) MHC Class II molecules in the face of one class II species (the transgenic species, for example HLA-DR) being blocked. This may be comparable to the effect in the target organism, where a mixture of MHC Class II molecules are expressed on the antigen presenting cells (APCs). For example, in humans, the “average” heterozygous human has several different class II molecules on their antigen presenting cells: two alleles of HLA-DR (with two different heterodimeric chains actually expressed in the context of each allele), two DQ alleles and two DP alleles. The details of the total number of class II molecules available on the APC will vary with different haplotypes, but for a typical person the APC may express around eight different class II molecules. Although a molecule such as the TDR31.1 antibody may be capable of binding to all human class II molecules, the affinity of interaction is likely to vary between different class II molecules. For example, TDR31.1 binds less strongly to HLA-DQ and HLA-DP than to HLA-DR. Thus, at least some class II molecules are likely to still be available for normal antigen presentation in the target organism, in a similar way as in a transgenic animal in which the host MHC class II molecules remain.

As will be apparent from the above, it is not necessary for both the β chain gene and the α chain gene to both be from the “target” organism, though this will generally be desirable. As noted above, some SAGs (eg SEB, SEC1-3, SED, TSST-1, SPEA) bind to the α chain of the MHC class II molecule whilst others bind to both the α and β chain (eg SEA, SEE) in which case it is clearly desirable that both chains in the model are from the target organism; and others may bind only to the β chain (eg SMEZ, SPEG, SPEH, SPEJ, SPEC). The α and/or β chain molecules may be chimaeric; for example the extracellular portion (or part therof) may be derived from the target organism (for example human) and the intracellular portion may be derived from the host organism (for example mouse). The skilled person will be able to select a suitable model for screening for agents in relation to a particular target organism and a particular superantigen or superantigens.

When the superantigen is a staphylococcal enterotoxin (SE) the cells usefully express a human HLA DR α chain and/or human HLA DR β chain.

When the superantigen is a streptococcal enterotoxin (SPE) the cells usefully express a human HLA DQ α chain and/or human HLA DQ β chain.

By this is included that at least part, preferably all, of the extracellular portion of the relevant chain is human.

There are transgenic animals, for example mice, which express particular transgenic T cells/T cell receptors, which makes them extra sensitive to particular superantigens. Such transgenic animals may be useful if they also are transgenic in relation to MHC class II molecules, for example if they were cross-bred with class II transgenic mice (as the class II appears to be the most important receptor).

In some circumstances it may be desirable to use as a transgene a particular MHC Class II molecule selected on the basis of frequency in a particular subpopulation or strain of mammal. For example, different MHC Class II alleles are found with different frequencies in different human populations and it may be desirable to select particular alleles depending on the population for which the agent is particularly intended. See, for example, Hill A. 1998. The immunogenetics of human infectious diseases. Annu. Rev. Immunol.; 16:593-617.

The cell or cells used in the method may be spleen cells, as described in Example 1 or in Sriskandan et al (2001) J Infect Dis 184, 166-1743. In particular the spleen cells may be from a transgenic animal, for example a transgenic mouse, as described above.

The method may measure the effect of the test compound on the superantigen-induced proliferation of cells, for example spleen cells (see, for example, Sriskandan et al (2001) supra). Such an assay can be conducted simply and efficiently using a 96 well plate format. A compound useful for prevention or amelioration of SAG poisoning is considered to reduce the cellular proliferation caused by the superantigen.

Other parameters (responses) that may be tested include expression of a particular cytokine (for example IL12, IL6, IFNγ or TNFα) or expression of a reporter gene, for example a reporter gene expressing a fluorescent substrate. For example, IL2 and NF-kappaB reporter gene systems are known and are suitable. Cell surface expression of markers of T cell activation, for example the IL2 receptor, may also be tested. Cells constitutively expressing a fluorescent protein may be useful in measuring cell proliferation, for example in a high-throughput assay.

Methods of determining whether a real or significant change, preferably reduction, of cellular proliferation (or other paramater, as noted above) has been achieved will be well known to those skilled in the art. This may be assessed using statistical methods or, particularly for initial screening of compounds, by selection of a cut-off point, with or without comparison with controls. Even if each test may not be compared with a control or the cut-off not adjusted by reference to a control, controls may still generally be included for validation of the test. Examples of types of control samples that may be used will be well known to those skilled in the art, and are also apparent from Example 1. For example, controls may include samples that are not exposed to compound; that are not exposed to superantigen; where transgenic cells are used, controls using cells which lack the transgenes. As examples, a 2-fold, 4-fold, 5-fold, 8-fold, 10-fold, 15-fold or 20-fold (or more) alteration in the parameter (for example cell proliferation, for example as measured by incorporation of tritiated thymidine, for example as in Example 1) may be achieved.

The test superantigen (or superantigens) may be a naturally occuring superantigen or may be a modified superantigen or superantigen fragment (for example as described in U.S. Pat. No. 5,859,207; US 2003/0009015; WO 96/14744; WO 03/002143WO 00/09154; U.S. Pat. No. 6,399,332). If the superantigen is not a full length native superantigen then it is desirable that the superantigen retains unmodified the regions considered to interact with the MHC Class II molecules (as discussed in the documents relating to modified superantigens or fragments). Superantigens may be obtained from Toxin Technology or Sigma or may be expressed recombinantly using standard methods.

The test conventional antigen may be purified protein derivative (PPD) of Mycobacterium tuberculosis. This may be obtained from Sigma. The reaction that is tested may again be cellular proliferation or measurement of cytokine release, as described in Example 1 and well known to those skilled in the art. Tetanus toxoid may also be used in human cells as a test conventional antigen. As discussed above in relation to tests in relation to SAG, methods of determining whether a real or significant change in the parameter is absent will be well known to those skilled in the art. As examples, less than a 1.2-fold, 1.4-fold, 1.5 fold, 1.8-fold, 2-fold or 3-fold alteration in the parameter (for example cell proliferation, for example as measured by incorporation of tritiated thymidine, for example as in Example 1) may be achieved.

The cells used for both the SAG and conventional antigen screen may be transgenic cells, or may be non-transgenic cells, for example peripheral blood monocytes (PBMCs). Transgenic cells may be useful in allowing tests to be extended to whole animals, but human cells may be useful for confirming effects in a human background.

Compounds, for example compounds selected using a cellular assay, may be tested in a whole animal in vivo screen, as described in Sriskandan et al (2001) supra. Compounds may be assessed in terms of lethality or in relation to histopathological changes or externally visible changes, as will be well known to those skilled in the art. For example, inflammatory infiltrates in upper and/or lower airways in response to respiratory exposure may be measured. Weight may be measured; weight loss (for example discernible over about 8 to 24 hours after exposure in the HLA-DR transgenic mice of Example 1) is associated with SAG action. Compounds may be assessed for their ability to prevent or ameliorate SAG poisoning when administered prior to the SAG or to prevent or ameliorate poisoning when administered after administration of the SAG.

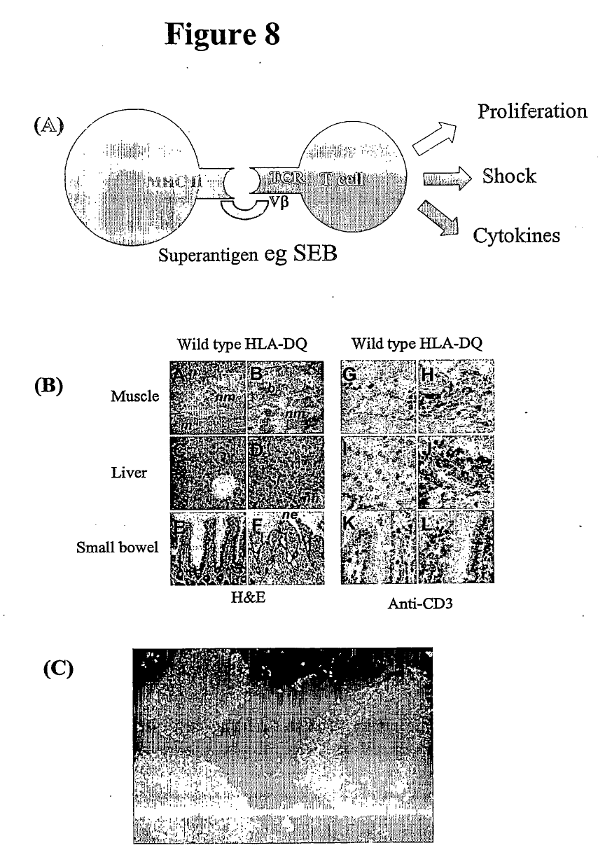

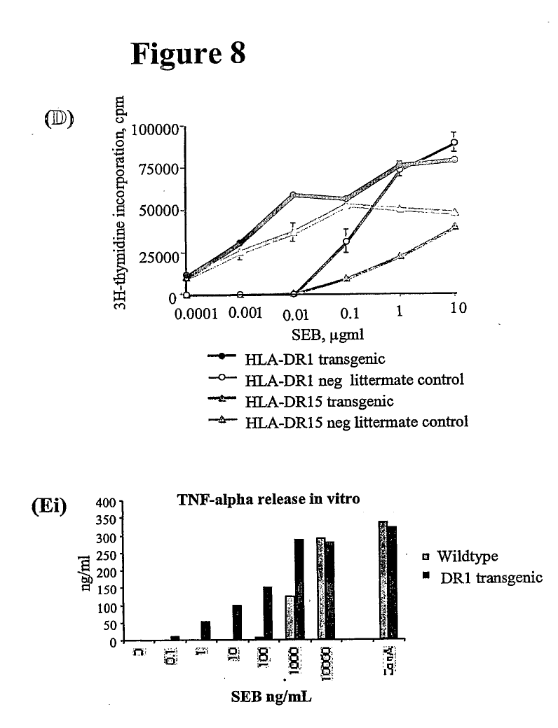

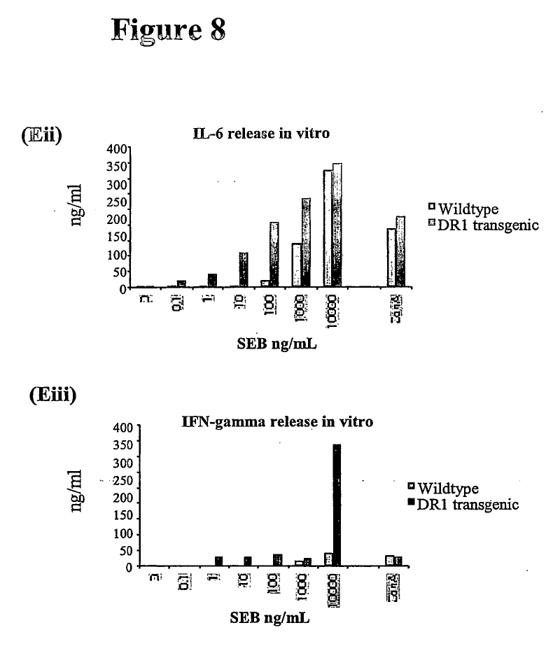

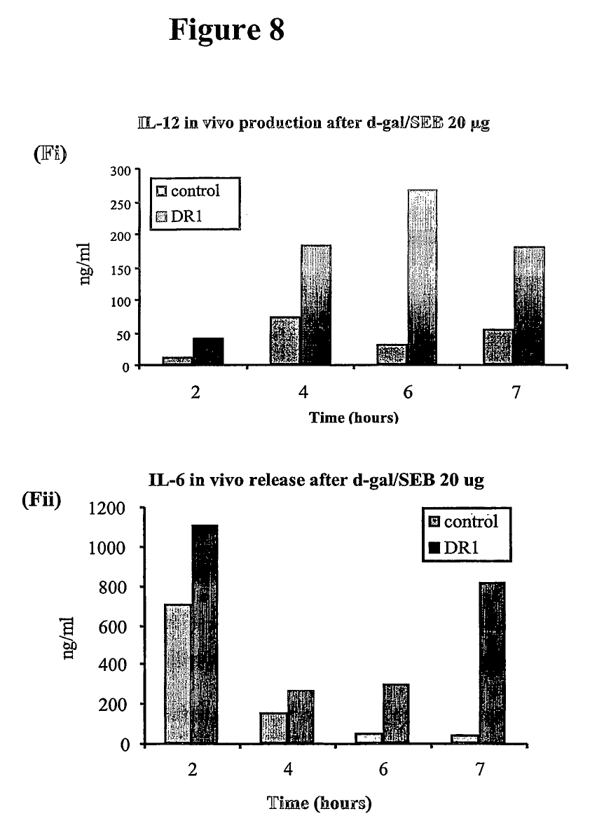

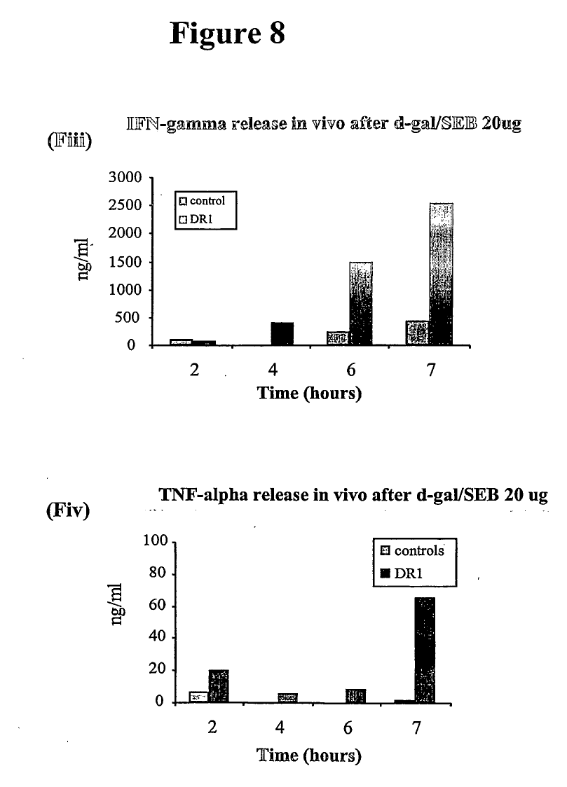

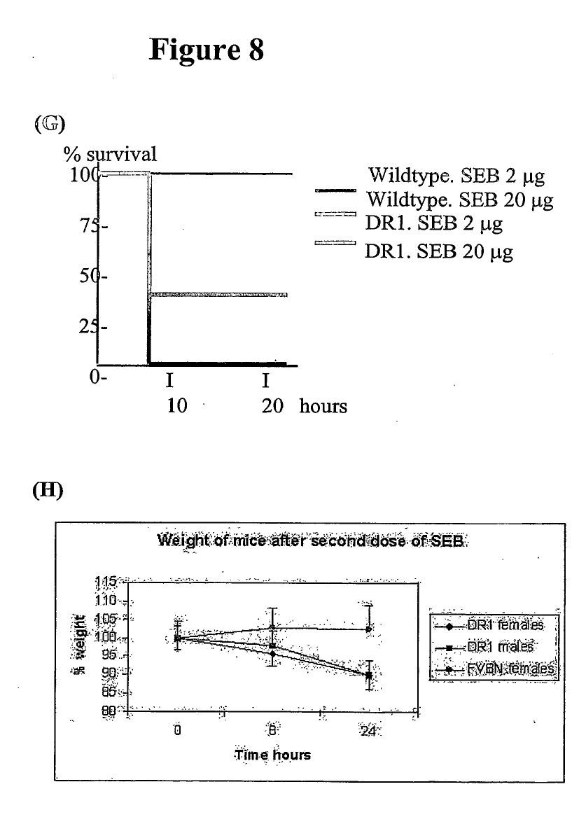

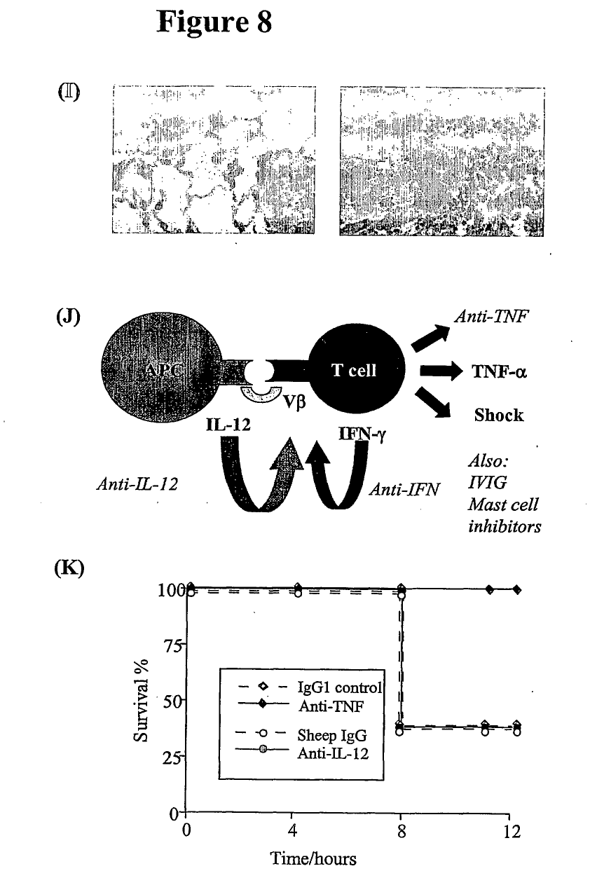

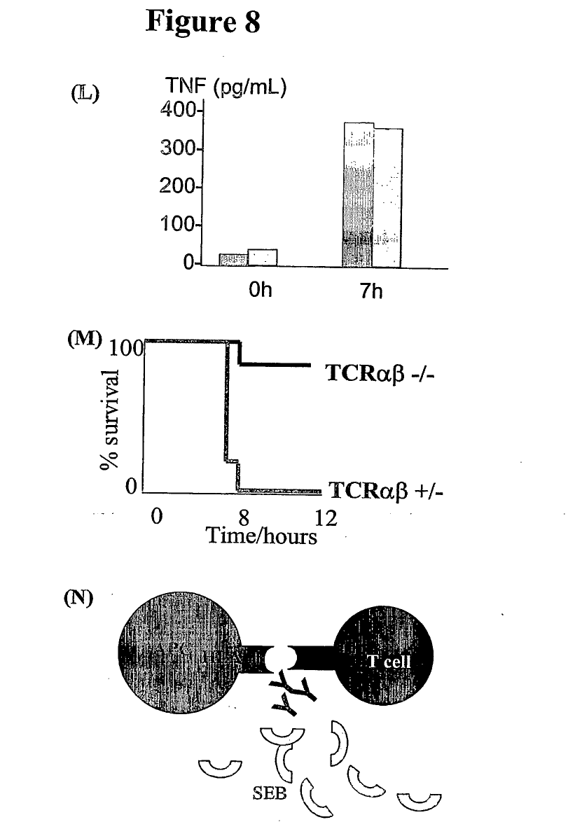

As example, the following in vivo methods can be used to screen potential therapeutic reagents for beneficial anti-superantigen activity in vivo: analysis of cytokine release in serum (in particular IL-6 as a recognised marker of disease severity) and/or survival, for example in d-galactosamine-pretreated HLA DR1 transgenic mice; analysis of weight loss, survival, and cytokine release, for example in HLA transgenic mice receiving a double dose of superantigen; analysis of weight loss, survival, cytokine release, and pulmonary inflammation (as can be measured by a number of recognised techniques including bronchial lavage and histopathology) in HLA transgenic mice receiving mucosally administered superantigen. Further information regarding model systems is given, for example, in Example 1 and FIG. 8.

A further aspect of the invention provides a method for providing a compound for test in a screening method of the first aspect of the invention comprising
(1)immunising an animal with a Class II MHC molecule from the first type of mammal as defined in relation to the first aspect of the invention (ie the target organism, for example human),
(2) obtaining immune serum from the animal for use as a test compound in a method of the first aspect of the invention; or
(3) Preparing a hybridoma using a splenocyte from the animal
(4) Optionally testing antibody produced by the hybridoma for ability to bind to the MHC Class II molecule
(5) Obtaining antibody expressed by the hybridoma (optionally which tests positive in step (3)) for use as a test compound in a method of the first aspect of the invention.

Methods of immunising animals and preparing hybridomas will be well known to those skilled in the art, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

In this context, by Class II molecule is included a portion of a full length Class II molecule, for example all or part of the extracellular domain of one or both chains of a full-length Class II molecule. The antigenic molecule used may be a recombinant polypeptide or may be a synthetic polypeptide, as well known to those skilled in the art. Peptides in which one or more of the amino acid residues are chemically modified, before or after the peptide is synthesised, may be used providing that the function of the peptide, namely the production of specific antibodies in vivo, remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the peptide from in vivo metabolism. The peptides may be present as single copies or as multiples, for example tandem repeats. Such tandem or multiple repeats may be sufficiently antigenic themselves to obviate the use of a carrier. It may be advantageous for the peptide to be formed as a loop, with the N-terminal and C-terminal ends joined together, or to add one or more Cys residues to an end to increase antigenicity and/or to allow disulphide bonds to be formed. If the peptide is covalently linked to a carrier, preferably a polypeptide, then the arrangement is preferably such that the peptide of the invention forms a loop.

According to current immunological theories, a carrier function should be present in any immunogenic formulation in order to stimulate, or enhance stimulation of, the immune system. It is thought that the best carriers embody (or, together with the antigen, create) a T-cell epitope. The peptides may be associated, for example by cross-linking, with a separate carrier, such as serum albumins, myoglobins, bacterial toxoids and keyhole limpet haemocyanin. More recently developed carriers which induce T-cell help in the immune response include the hepatitis-B core antigen (also called the nucleocapsid protein), presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both. Alternatively, several copies of the same or different peptides of the invention may be cross-linked to one another; in this situation there is no separate carrier as such, but a carrier function may be provided by such cross-lining. Suitable cross-linking agents include those listed as such in the Sigma and Pierce catalogues, for example glutaraldehyde, carbodiimide and succinimidyl 4-(N-maleimidomethyl)cyclohexane-1-carboxylate, the latter agent exploiting the —SH group on the C-terminal cysteine residue (if present).

If the peptide is prepared by expression of a suitable nucleotide sequence in a suitable host, then it may be advantageous to express the peptide as a fusion product with a peptide sequence which acts as a carrier. Kabigen's “Ecosec” system is an example of such an arrangement.

A further aspect of the invention provides a method of treating an individual with or at risk of superantigen (for example SEB) exposure/poisoning comprising administering to the individual the antibody TDR31.1 or molecule having the binding specificity of TDR31.1.

A further aspect of the invention provides the use of antibody TDR31.1 or molecule having the binding specificity of TDR31.1 in the manufacture of a medicament for treating an individual with or at risk of superantigen (for example SEB) exposure/poisoning.

A further aspect of the invention provides a method of treating an individual with or at risk of superantigen (for example SEB) exposure/poisoning comprising administering to the individual a molecule identified using the screening method of the invention.

A further aspect of the invention provides the use of a molecule identified using the screening method of the invention in the manufacture of a medicament for treating an individual with or at risk of superantigen (for example SEB) exposure/poisoning.

The exposure or risk of exposure may arise from biowarfare or bioterrorism. The methods of treatment or medicaments of the present invention may be particularly useful in relation to exposure arising from biowarfare or bioterrorism because of the increased importance of MCH Class II interactions in such exposure.

The aforementioned compounds of the invention or a formulation thereof may be administered by any conventional method including oral, inhaled and parenteral (eg subcutaneous or intramuscular) injection. The treatment may consist of a single dose or a plurality of doses over a period of time.

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

Proteins and peptides may be delivered using an injectable sustained-release drug delivery system; this may be appropriate for some individuals, for example in the context of prophylactic treatment or the treatment of autoimmune disease. These are designed specifically to reduce the frequency of injections that may otherwise be required. An example of such a system is Nutropin Depot which encapsulates recombinant human growth hormone (rhGH) in biodegradable microspheres that, once injected, release rhGH slowly over a sustained period.

The protein and peptide can be administered by a surgically implanted device that releases the drug directly to the required site. Proteins and peptides can be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Protein and peptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

The compound may be delivered alongside other treatment for SAG poisoning, for example as discussed in Example 2.

The TDR31.1 antibody or other compound may also be useful in the treatment of patients with autoimmune disease. Accordingly the invention provides a method of treating an individual with or at risk of an autoimmune disease or condition comprising administering to the individual the antibody TDR31.1 or molecule having the binding specificity of TDR31.1.

The invention further provides a method of treating an individual with or at risk of an autoimmune disease or condition comprising administering to the individual a molecule identified using the screening method of the invention.

All references mentioned herein are hereby incorporated by reference.

The invention is now described in more detail by reference to the following, non-limiting Figures and Examples.

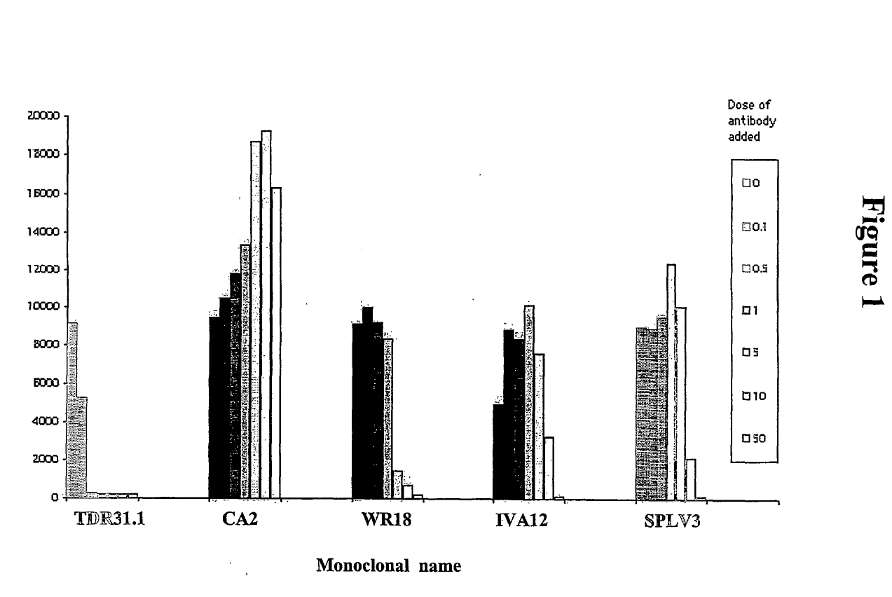

FIG. 1: Effect of different anti-HLA-class II monoclonals on SEB-induced DR1 transgenic spleen cell proliferation

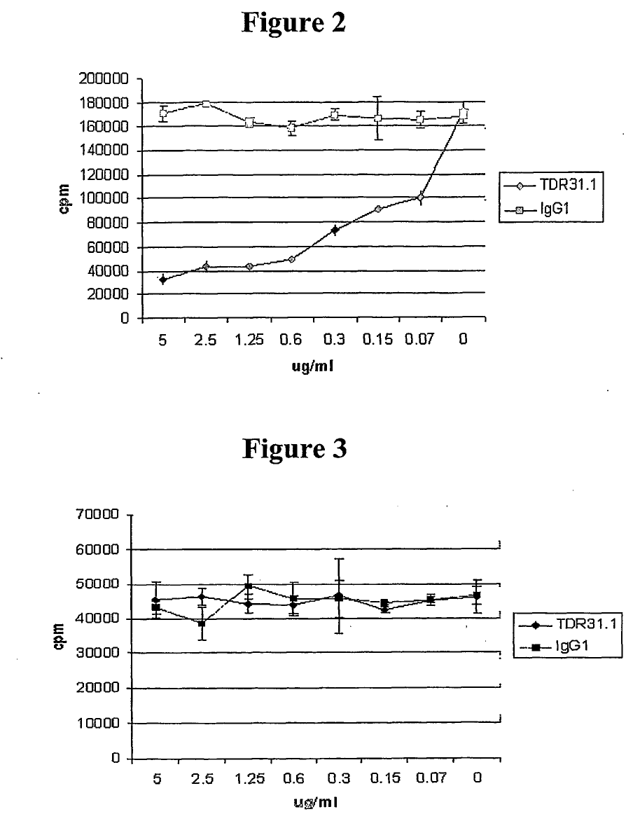

FIG. 2: Effect of TDR31.1 antibody on SEB induced proliferation of lymph node cells from a DR1 mouse

FIG. 3: Effect of TDR31.1 antibody on PPD induced proliferation of lymph node cells from a DR1 mouse

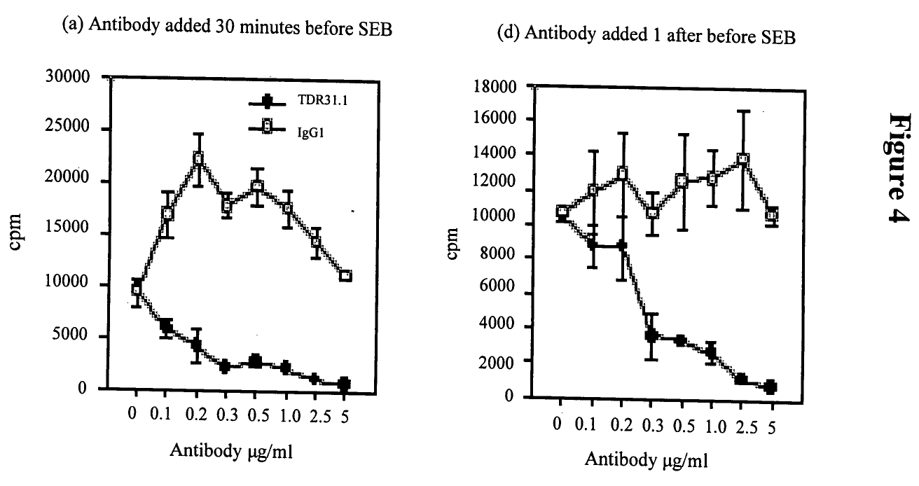

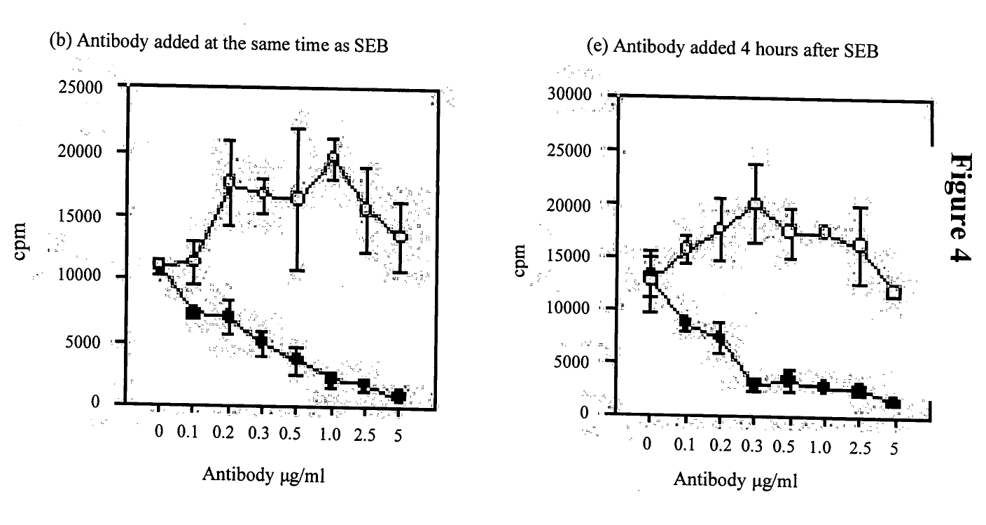

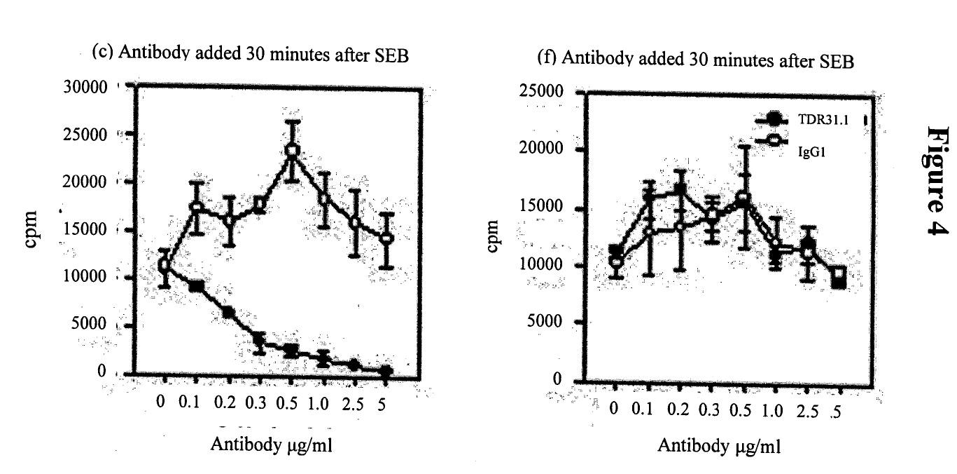

FIG. 4: Effect of TDR31.1 antibody added at different times relative to SEB

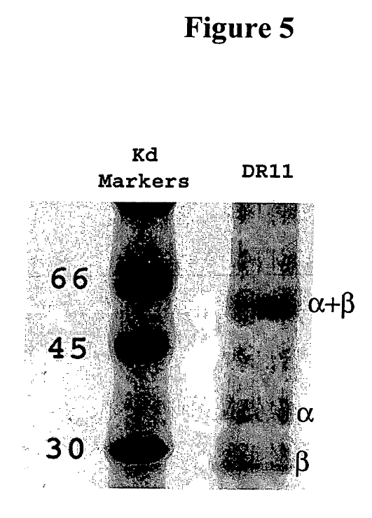

FIG. 5: Expression of HLA-DR heterodimers by B lymphoblastoid cell lines

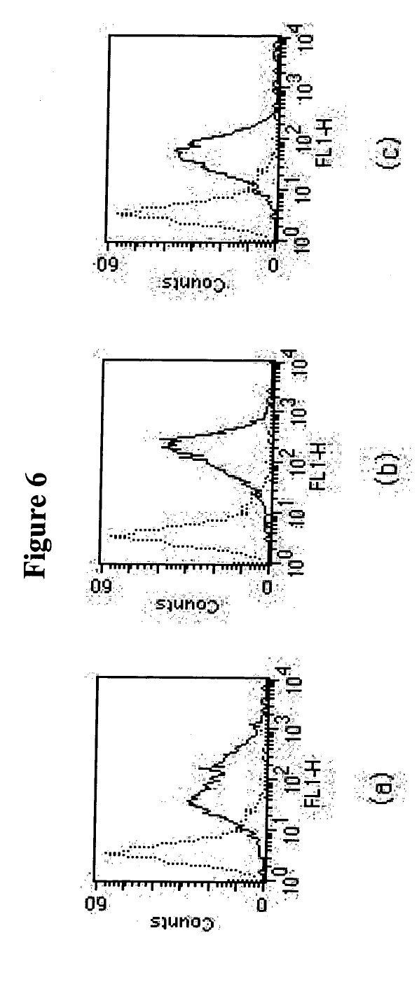

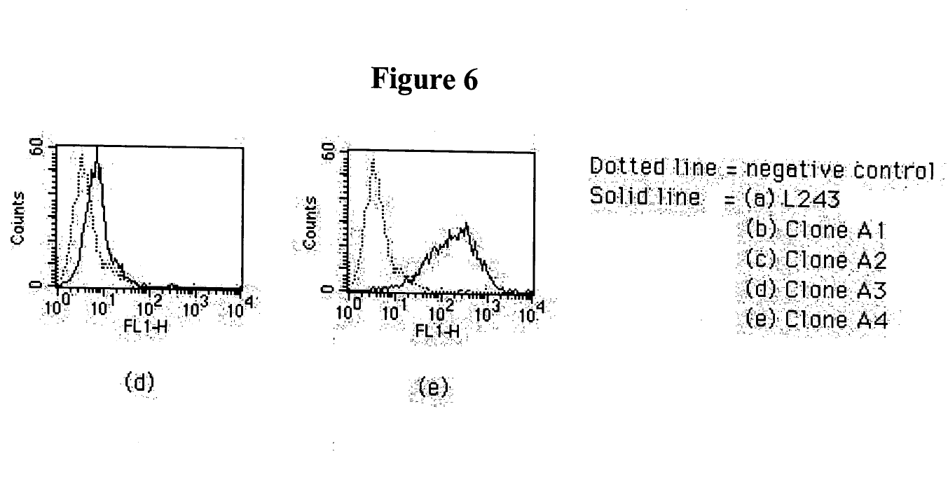

FIG. 6: FACS analysis of hybridomas. Clones A1, 2 and 4 are positive for HLA-DR staining

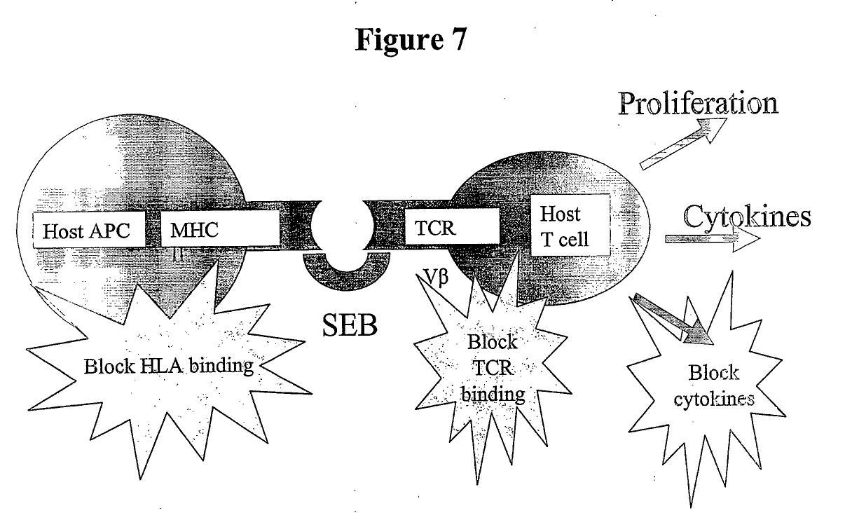

FIG. 7: Strategies for blocking the effects of superantigens

FIG. 8: Trangenic disease models to develop superantigen countermeasures

A. The staphylococcal enterotoxins (SEs) are identified as potential biowarfare and boterrorism agents. Acting as superantigens in vitro, they can cause severe emesis and diarrhea if ingested, and in primates can lead to respiratory distress and circulatory collapse if inhaled. SEs are easy to clone and express, are active at femtomolar-picomolar amounts in vitro, and can be lethal at microgram quantities in vivo. Sensitivity to SEs requires binding of toxin to the MHC class II molecule and cross linking of certain T cell receptor Vb subunits.

The aim of this project was develop and characterize an HLA-class II transgenic (tg) model of SEB shock in which to develop SE countermeasures using mice expressing HLA-DR1 or HLA-DR15. Pathogenic mechanisms were explored using neutralizing antibodies and by deletion of the ab T cell receptor (TCR).

B. Previous work had identified HLA-DQ transgenics which were particularly sensitive to the streptococcal toxin SPEA, compared with wild type mice. Immunohistochemistry showed T cell infiltrates in peri-portal regions and other tissues of HLA-DQ mice infected with a pathogen.

C. To study SEs, we employed HLA-DR transgenics; immunohistochemistry showed faithful tissue distribution of HLA-DR (spleen shown).

D and E. In vitro results. Transgenic mice were three logs more sensitive to SEB than wildtype mice, with regard to both T cell proliferation and cytokine response in vitro.

F and G. In vivo results-with d-galactosamine Cytokine release (F) and mortality (G) is enhanced by SEB in humanized trangenic mouse. DR1 transgenics are more susceptble to death than E. coli than are other strains.

H and I. In vivo results-without d-galactosamine In vivo, respiratory exposure to SEB induced inflammatory infiltrates within upper and lower airways. HLA-DR1 tg mice were 2-3 logs more sensitive to SEB than wildtype mice, with regard to both cytokine production (IL-12, IFN, TNF, IL-6), AST levels, and mortality. (H) Weight loss is seen after two doses of SEB in transgenics only. (I) Upper airways: perivascular infiltrates (left panel). Lower airways: mixed patchy infiltrate, mainly perivascular (right panel).

J to M. Application: Pathogenesis. Anti-TNF protected transgenic mice, in contrast to anti-IL12, or control antibodies (K). HLA-DR transgenic mice were then used, which had a mutation in the TCR alpha chain, reducing the number of circulating T cells. Signalling through the TCR appeared critical for mortality (M). However, TNF levels were unchanged by this mutation (L), showing that, in this model, the source of TNF is unlikely to be from T cells.

N. Application: Countermeasures. A number of strategies focus on molecules which bind the toxin, or which mimic the toxin in some way. We have accumulated evidence which indicates that the interaction with HLA class II is a key step. To develop broadly-specific reagents acting at the class II molecule, we have screened panels of existing and newly generated HLA class II monoclonals for inhibitory activity.

HLA-DR heterodimers were purified from B lymphoblastoid cell lines, then used to immunize mice, prior to developing panels of hybridomas reactive with HLA-DR+ cell lines (FIG. 6): a) staining with control mAb L243; b) staining with clone A1; c) staining with clone A2.

Screening has revealed at least one antibody (blue line) with potent SEB-inhibitory effects, compared with control IgG1 (pink line) (FIG. 2). The antibody had no adverse effect on PPD-induced T cell responses from PPD-immunized transgenic mice. The SEB inhibitory activity can be observed up to 4 hours after exposure of cells to SEB (FIG. 4) suggesting this may be a promising reagent for therapy.

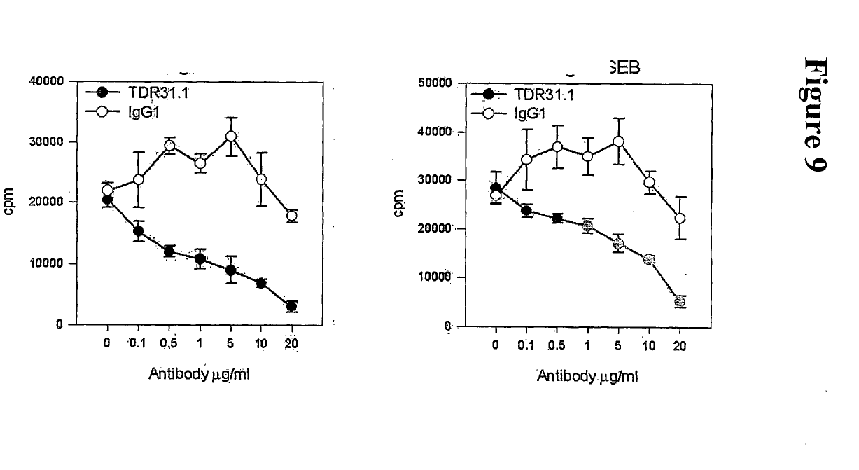

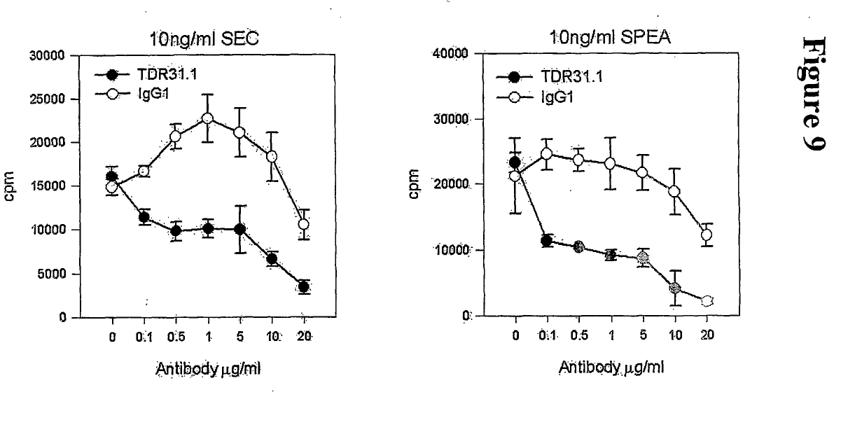

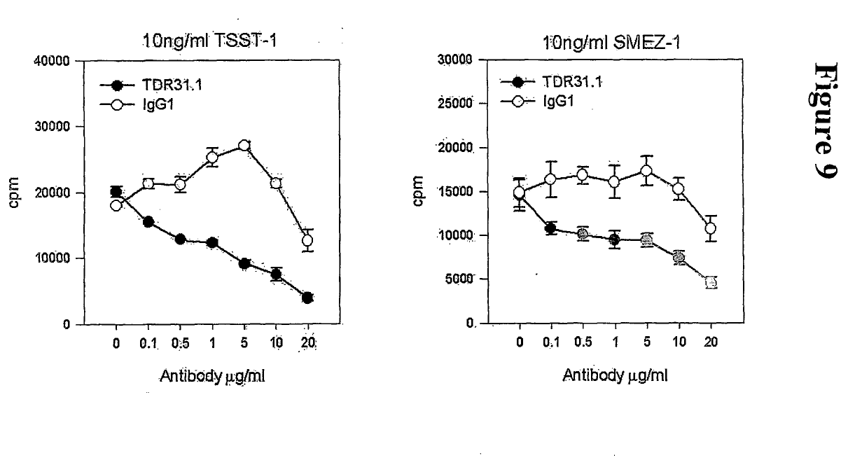

FIG. 9: The effect of TDR31.1 on the proliferative response of human PBL to different superantigens

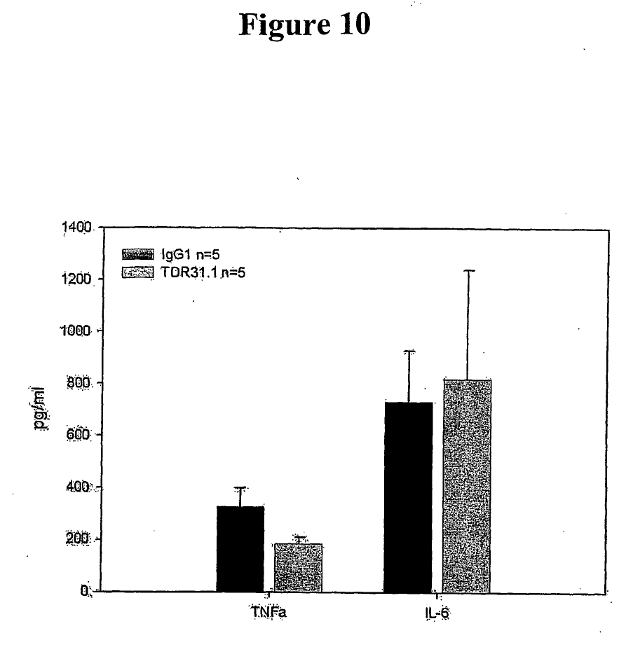

FIG. 10: Reduction in TNFα following TDR treatment.

Serum cytokines 6 hours after treatment of DR1 mice with 20 μg antibody followed by 20 mg Dga1 and 2 μg SEB. In further in vivo experiments, using five transgenic mice per group, TDR31.1 treatment significantly reduced levels of TNF-alpha produced in response to a challenge with SEB 2 μg by almost 50%. TNF-alpha is known to be a key mediator of death in this model of superantigen shock, as treatment with anti-TNF monoclonal is protective. Note that IL-6 levels do not reach the same magnitude in this experiment, presumably because the dose of SEB is lower.

FIG. 11: TDR31.1 recognises the β chain of HLA-DQ and HLA-DR

In contrast antibody L243 mainly recognises the α chain. HLA class II heterodimers were purified and run on SDS PAGE prior to immunoblotting with either L243 or TDR31.1.

### EXAMPLE 1

### Molecular Reagents for Reducing or Preventing the Deleterious Effects of the Immune Response to Superantigens

In order to identify molecular reagents for reducing or preventing the deleterious effects of the immune response to a broad range of superantigenic toxins produced by pathogenic bacteria we have analysed the effect of HLA class II blocking antibodies using in vitro and in vivo models. We have also investigated the ability of immunomodulators to reverse the effects of superantigen immunostimulation. We have identified molecules that may be useful as countermeasures to protect against the adverse effects of superantigens, particularly mucosal exposure to superantigenic exotoxins.

The overall aim of the study was to develop novel molecular reagents to reduce or prevent the deleterious effects of the immune response to a broad range of superantigenic toxins produced by pathogenic bacteria. The aim was to disrupt the interaction between HLA class II molecules, superantigens, and T cell receptors, an interaction which leads to the features of lethal toxic shock. The efficacy of the immunomodulatory agents are tested in an HLA class II transgenic mouse model that we have developed.

The molecules that we have developed are considered particularly suitable for use in the bioterrorism context. For example, in the bioterrorism context there is mucosal exposure (rather than infective toxic shock), in which we have found that the toxins appear to have toxic effects on the body independent of T cells—the time course is too rapid to be accounted for by conventional understanding of SAG-T cell interactions—the first receptor likely to interact would therefore be the class II molecule. Further, the molecules are considered to provide a short-lived but rapid effect, creating a transient state of protection rather than a long-lived (but slow-to-start) vaccine-based effect).

Further, we have provided a fully validated and characterized transgenic mouse model of superantigen-mediated disease, that can be used to study both pathogenic mechanisms and possible prophylactic or therapeutic countermeasures. We have demonstrated the ability of a HLA class II blocking antibody to prevent or ameliorate SEB-induced shock in our animal model.

Background. The staphylococcal enterotoxins (SEs) are identified as potential biowarfare and bioterrorism agents. Acting as superantigens in vitro, they can cause severe emesis and diarrhea if ingested, and in primates can lead to respiratory distress and circulatory collapse if inhaled. SEs are easy to clone and express, are active at femtomolar-picomolar amounts in vitro, and can be lethal at microgram quantities in vivo. Methods The aim of this project was to develop and characterize an HLA-class II transgenic (tg) model of SEB shock in which to develop SE countermeasures using mice expressing HLA-DR1 or HLA- DR15. Pathogenic mechanisms were explored using neutralizing antibodies and by deletion of the αβ T cell receptor (TCR). Results Tg mice were three logs more sensitive to SEB than wildtype mice, with regard to both T cell proliferation and cytokine response in vitro. In vivo, respiratory exposure to SEB induced inflammatory infiltrates within upper and lower airways. HLA-DR1 tg mice were 2-3 logs more sensitive to intraperitoneal (i.p.) SEB than wildtype mice, with regard to both cytokine production (IL-12, IFN, TNF, IL-6), AST (liver transaminases) levels, and mortality. Treatment with anti-TNF was protective whereas anti-IL-12 was not. Knockout mice lacking TCRαβ+ cells were also protected from death although, remarkably, TNF levels were unaltered, demonstrating that T cells are unlikely to be the main source of TNF in this model. Targeting the interaction of SE with antigen presenting cells, large-scale screening of antibodies with potential broad spectrum anti-SE activity has already identified one monoclonal (TDR31.1) which inhibits SEB in vitro but, importantly, does not inhibit responses to PPD (purified protein derivative) from Mycobacter tuberculosis. Conclusions. The HLA class II tg mouse is now an established model for investigation of superantigen pathogenesis. The use of TCR knockouts will identify the precise role played by T cells in mucosal exposure to SEs, and, together with array analysis, will enable therapeutic targets to be more accurately predicted. See also FIG. 8

Screening of Existing Anti-HLA Class II Antibodies

A panel of >12 anti-HLA class II (DQ and DR) hybridoma supernatants were purified by protein G column chromatography. In vitro SEB-class II inhibitory action of the antibodies was evaluated using SEB-stimulated spleen cells from HLA-DR1 transgenic mice. Measurement of cellular proliferation (by incorporation of tritiated thymidine) demonstrated that some antibodies (e.g. TDR31.1) have significant SEB-inhibitory activity, even at low concentrations (0.1-0.5 μg/mL, see illustration). Higher concentrations of antibody (50 μg/mL) have non-specific inhibitory effects, as seen with the anti-HLA-DQ antibody (SPLV3), which would not be expected to inhibit the interaction between SEB and HLA-DR. We have therefore demonstrated that inhibition of superantigen-induced immune cell activation can be blocked by certain anti-class II antibodies.

Using in vitro analysis, we have now established that TDR31.1 does not inhibit cognate responses to antigen, and therefore should not pose an immunosuppressive risk to the host. Specifically, we have confirmed that the antibody has superantigen-specific inhibitory effects on T cell stimulation which do not adversely impact upon antigen-specific processing, presentation, and T cell response, in the following way: Using HLA-DR1 transgenic mice which had been immunized with the purified protein derivative (PPD) of M. tuberculosis, murine T cell responses to either PPD or SEB were analysed ex vivo in the presence of different concentrations of TDR31.1. In order to fully appreciate the potential effects of the antibody on PPD (and SEB-)-induced responses, TDR31.1 was co-incubated with the cell suspension for 30 minutes prior to addition of either PPD or SEB. Under these conditions, the monoclonal antibody inhibited SEB-induced responses, but did not adversely affect PPD-induced responses (which were of lesser magnitude). An isotype control antibody had no effect in either setting.

To further characterize the in vitro effects of TDR31.1 on SEB-induced proliferation of T cells, with specific regard to timing of addition, TDR31.1 was added to T cells either at the same time as SEB or at 0.5 h, 4 h. or 24 h after SEB addition. The data showed that the inhibitory effects of TDR31.1 persist, when the reagent is added at −0.5, 0, 0.5, or 4 h with respect to addition of SEB to T cells (see FIG. 4). Delaying the addition of antibody for 24 h was not compatible with inhibition, a result which is predictable from the kinetics of TCR signalling and cytokine transcription by this timepoint. Nevertheless, the inhibition at times after SEB exposure is particularly useful with regard to the post-exposure treatment for SEB-induced shock.

The in vitro experiments were conducted using an SEB concentration of 10 ng/mL, which routinely gives strong T cell proliferative responses in vitro in the transgenic setting. If the concentration of SEB Were as high as 10 μg/ml SEB, preliminary studies show that 5 μg/mL antibody will be needed to achieve 50% inhibition.

We have also shown that administration of a dose of antibody TDR31.1 which would partially inhibit all superantigenic activity in vivo was of benefit in d-galactosamine sensitized mice receiving a lethal dose of SEB: IL-6 levels were significantly reduced from 1168.4±191 pg/mL in mice receiving control IgG1 reagent, to 760.8±222 pg/mL in mice receiving the test antibody (p value 0.028, Mann Whitney U test).

New “Customized” Anti-HLA Class II Antibodies for Blocking Superantigens

Since pre-existing HLA monoclonals have been screened and selected on the basis of their use in HLA typing (and therefore probably for specificity for a particular HLA type) and not specifically for superantigen blocking, antibodies with this property would not have been retained in existing panels of monoclonals. We therefore commenced a study to generate a customized monoclonal antibody, in which mice were hyperimmunised (ie with repeat immunisations) with affinity-purified HLA-DR heterodimer for the stimulation of high titre HLA antibodies. HLA-DR heterodimers were purified from large volume cultures of B lymphoblastoid cell lines which express HLA-DR (see FIG. 5).

To assess the titer of antibodies in immune mice prior to fusion, immune sera were screened for ability to bind to HLA-DR-expressing cells by FACS. The hyperimmune sera were titrated for binding to HLA-DR molecules on the DR-positive cell line, IDF, or on the bare lymphocyte cell line, BLS, as a negative control. Sera contained high titers of specific DR-binding antibodies. Splenocytes from these mice were then fused with the Sp1 fusion partner to generate hybridomas which are screened for the specific inhibition of bacterial superantigen presentation to T cells.

To identify the presence of antibody capable of binding HLA-DR positive B cell lines, screening of hybridoma-positive wells by FACS analysis was performed. 134 out of 480 hybridoma supernatants screened have shown positive fluorescent staining of HLA-DR positive B cell lines, compared to non specific immunoglobulin staining (as a negative control) and the pan-DR monoclonal L243 (as a positive control). In FIG. 6, clones A1, 2, and 4 are positive for HLA-DR staining.

To select the monoclonals with greatest SEB inhibitory activity, monoclonals are coincubated with SEB-stimulated spleen cells from the transgenic mice, as described earlier. Preliminary studies have shown that un-purified hybridoma supernatants contain growth factors which interfere with the SEB-induced T cell proliferation assay; it is therefore necessary to purify immunoglobulin fractions from DR-reactive hybridoma supernatants prior to testing for biological activity.

Development of Small Molecules with Similar Activity

Antibody TDR31.1 recognises a non-polymorphic class II beta chain epitope of the HLA class II molecule and is reputed to recognise HLA DP, DQ, and DR. To our knowledge, formal epitope mapping has not been performed. Given that all superantigens are considered to interact with either/both HLA-DR or HLA-DQ, it is expected that TDR31.1 has inhibitory action against all/a very wide range of superantigens.

SEB is known to bind the alpha chain of class II molecules, whilst TDR31.1 binds the beta chain. We therefore consider that TDR31.1 is unlikely to be acting as a simple competitive inhibitor of SEB binding. For this reason, we consider that TDR31.1 will have inhibitory action against other superantigens which bind in alternative positions on the class II molecule.

The panel of ‘customized’ anti-HLA-DR monoclonals is likely to contain one or more monoclonals with equivalent or improved activity compared with TDR31.1. To develop alternative molecules with similar inhibitory activity, the active monoclonal antibody or antibodies will be formally epitope mapped (for example by incubation of monoclonal antibody or antibodies with peptides spanning the relevant class II structure, using a robotic peptide synthesizer; or using known techniques for identifying epitopes or mimotopes, for example using an “epitope library”; see, for example, Scott & Smith (1990) Science 249, 386-390 and WO 97/18236 and references therein). The identified epitope/amino acid sequences would then be used to design or select small molecules (which may be a peptide or peptidomimetic compound) which would be predicted to bind to the identical region of the class II structure. These molecules would be screened initially for binding to peptides generated by the robotic peptide synthesizer, and also by an in vitro biological assay, for their ability to inhibit SEB (or other superantigen)—induced proliferation of spleen cells, an assay which can be conducted simply and efficiently using a 96 well plate format.

### EXAMPLE 2

### Treatment of an Individual with an MHC Class II-Modulating Compound Such as TDR31.1 Antibody

The individual may be an individual (preferably a human individual) at imminent risk of superantigen exposure/poisoning (for example about to enter a war zone or zone at risk of biowarfare/bioterrorism attack) or suffering from superantigen exposure/poisoning (for example as a consequence of biowarfare/bioterrorism attack). The MHC Class II-modulating compound (for example TDR31.1 antibody) is administered to the patient, for example at about 20-40 mg intravenously. Guidance on treatment using monoclonal antibodies is provided in, for example, Coles et al (1999) Lancet 354, 1691-1695, which discusses treatment using the Campath-1HT™ monoclonal antibody. The MHC Class II-modulating compound may be administered alongside other treatments, for example anti-TNF treatment and/or intravenous fluids, vasopressors and possibly also intravenous immunoglobulin (IVIG) to the superantigen (if known); see McCormick et al (2001) Annu Rev Microbiol 55, 77-104 for a review. If the individual has already been exposed to the superantigen then the MHC Class II-modulating compound is administered as soon as possible (in order of preference within about 12, 8, 6 or 4 hours of exposure). The MHC Class II-modulating compound may be supplied to the individual for emergency use if symptoms are experienced, for example in a tablet form or inhaler form or in an injectable format (similar to a “combopen” as used for the anti-nerve agent atropine).

### EXAMPLE 3

### Effect of TDR31.1 on the Proliferative Response of Human PBL to Different Superantigens

The TDR31.1 antibody is inhibitory for the proliferative response of human PBMCs for a range of superantigens, in contrast to the isotype control. The TDR31.1 antibody did not, however, inhibit the human PBMC responses to PPD, a conventional antigen (not shown).

### EXAMPLE 4

### Reduction in TNFα Following TDR Treatment

In in vivo experiments, using five transgenic mice per group, TDR31.1 treatment significantly reduced levels of TNFα produced in response to a challenge with SEB 2 μg by almost 50%. TNFα is known to be a key mediator of death in this model of superantigen shock, as treatment with anti-TNF monoclonal is protective.

The IL-6 levels do not reach the same magnitude in this experiment as in that reported in Example 1, which is considered to be because the dose of SEB is lower.

1-19. (canceled)

20. A method for identifying a compound for modulating the immune response in a first type of mammal to a superantigen, comprising steps of:
(1) providing a cell expressing a MHC Class II molecule derivable at least in part from said first type of mammal or cells comprising such a cell or cells;
(2) exposing the cell or cells provided in step (1) to a test compound;
(3) exposing said cell or cells to said superantigen selectively before, after or during step (2);
(4) determining whether said test compound modulates a response of said cell or cells to exposure to a superantigen;
(5) optionally, determining whether said test compound modulates a response of such a cell or cells to exposure to a conventional antigen; and
(6) selecting a compound which modulates a response to the superantigen and does not modulate the response to exposure to the conventional antigen.

21. A method as in claim 20 wherein the test compound is selected from the group consisting of monoclonal antibodies reactive with MHC Class II molecules and molecules having the binding specificity of a monoclonal antibody reactive with an MHC Class II molecule.

22. A method as in claim 21 wherein the monoclonal antibody is reactive with the MHC Class II molecule as defined in claim 20 or a part thereof.

23. A method as in claim 20 wherein said cell expressing a MHC Class II molecule derivable at least in part from the first type of mammal is a cell of a second type of mammal which expresses at least part of an MHC Class II molecule of said first type of mammal.

24. A method as in claim 23 wherein the second type of mammal is mouse and the first type of mammal is human.

25. A method as in claim 23 wherein the cell also expresses a MHC Class II molecule of the second type of mammal.

26. A method as in claim 20 wherein the cell or cells comprising such a cell is/are spleen cells.

27. A method as in claim 26 wherein the spleen cells are from a transgenic mouse.

28. A method as in claim 20 wherein said cells express a human HLA DR and/or HLA DQ MHC Class II molecule or part thereof.

29. A method as in claim 20 wherein said response is cellular proliferation.

30. A method as in claim 29 wherein the compound reduces the cellular proliferation caused by the superantigen.

31. A method as in claim 28 wherein when the superantigen is a staphylococcal enterotoxin (SE), the cells express a human HLA DR α chain and/or human HLA DR β chain.

32. A method as in claim 28 wherein when the superantigen is a streptococcal enterotoxin (SPE), the cells express a human HLA DQ α chain and/or human HLA DR β chain.

33. A method for providing a compound for test in a method according to claim 20 comprising steps of:
(1) immunizing an animal with a MHC Class II molecule from said first type of mammal as defined in claim 20;
(2) selectively obtaining immune serum from the animal for use as a test compound in said method of claim 20, or preparing a hybridoma using a splenocyte from the animal;
(3) optionally testing antibody produced by the hybridoma for ability to bind to the MHC Class II molecule;
(4) obtaining antibody expressed by the hybridoma (optionally which tests positive in step (3)) for use as a test compound in said method of claim 20.

34. A method of treating an individual with or at risk of a condition selected from the group consisting of superantigen exposure/poisoning or an autoimmune disease or condition comprising a step of administering to the individual an entity selected from the group consisting of the antibody TDR31.1, a molecule having the binding specificity of TDR31.1 and a molecule identified using the method of claim 20.

35. A method as in claim 24 wherein the cell or cells comprising such a cell is/are spleen cells.

36. A method as in claim 36 wherein the spleen cells are from a transgenic mouse.
